(19) United States
(12) Patent Application Publication
(10) Pub. No.: US 2026/0182966 A1
(43) Pub. Date: Jul. 2, 2026

(54) INTELLIGENT MEDICAL IMAGING PARAMETER RECOMMENDATION TOOL

(71) Applicant: GE Precision Healthcare LLC, Waukesha, WI (US)

(72) Inventors: Mostafa Shahriari Shourabi, Linz (AT); Stephan Anzengruber, Ried imInnkreis (AT)

(73) Assignee: GE Precision Healthcare LLC, Waukesha, WI (US)

(21) Appl. No.: 19/006,887

(22) Filed: Dec. 31, 2024

(51) Int. Cl. A61B 8/00

(52) U.S. Cl. CPC A61B 8/5207; A61B 8/5223; A61B 8/5269; A61B 8/54

(57) ABSTRACT

An artificial intelligence-assisted ultrasound imaging parameter recommendation tool is disclosed. In one example, an ultrasound imaging system includes a processing circuit having a processor coupled to a memory device storing instructions thereon that, when executed, cause the processing circuit to perform operations including identifying contextual information regarding a patient; determining, based on the contextual information, a plurality of sets of imaging parameters; receiving initial image data of an anatomical region obtained using the plurality of sets of imaging parameters; presenting the plurality of initial images based on the initial image data; receiving a user input selecting a preferred image of the plurality of images, the preferred image having been obtained using a preferred set of imaging parameters; and configuring an ultrasound probe to receive additional image data of the anatomical region using the preferred set of imaging parameters.

### FIELD

Embodiments of the subject matter disclosed herein relate to ultrasound imaging, and more particularly, to providing recommendations of ultrasound imaging parameters based on contextual information regarding an ultrasound scan.

### BACKGROUND

During a medical imaging scan, a plurality of medical images of a patient are obtained by a technician, such as a sonographer, to measure or detect various aspects of anatomical features present within the medical images. Acquisition parameters used to obtain the medical images (e.g., a frequency, acquisition angle, dynamic power, gain, compound imaging, etc.) impact the resulting image quality of the medical images and are therefore chosen by the technician to optimize the image quality.

### SUMMARY

An embodiment relates to an ultrasound imaging system. The ultrasound imaging system includes a transducer configured to transmit and receive an ultrasound signal, a matching layer configured to have an acoustic impedance between a tissue to be imaged and a material of the transducer, a damping block configured to absorb ultrasound energy, and a processing circuit. The processing circuit includes a processor coupled to a memory device storing instructions thereon that, when executed, cause the processing circuit to perform operations including identifying contextual information regarding a patient of an ultrasound scan. The operations include determining, using a machine learning model and based on the contextual information, a plurality of sets of imaging parameters. The operations include receiving initial image data obtained by the transducer and of an anatomical region obtained using the plurality of sets of imaging parameters, wherein the initial image data is obtained with an ultrasound probe in a fixed position over the anatomical region. The operations include presenting a plurality of initial images based on the initial image data on a display device of the ultrasound imaging system, wherein each of the initial images correspond to one set of imaging parameters from among the plurality of sets of imaging parameters. The operations include receiving a user input selecting a preferred image from among the plurality of initial images, the preferred image having been obtained using a preferred set of imaging parameters of the plurality of sets of imaging parameters. The operations include configuring the ultrasound probe to receive additional image data from the transducer and of the anatomical region using the preferred set of imaging parameters.

Another embodiment relates to a medical imaging system including a processing circuit having a processor coupled to a memory device storing instructions thereon that, when executed, cause the processing circuit to perform operations. The operations include identifying contextual information regarding a patient of a medical imaging scan. The operations include determining, using a machine learning model and based on the contextual information, a plurality of sets of imaging parameters. The operations include receiving initial image data of an anatomical region obtained using the plurality of sets of imaging parameters, wherein the initial image data is obtained with an ultrasound probe in a fixed position over the anatomical region. The operations include presenting a plurality of initial images based on the initial image data on a display device of the medical imaging system, wherein each of the initial images correspond to one set of imaging parameters from among the plurality of sets of imaging parameters. The operations include receiving a user input selecting a preferred image from among the plurality of initial images, the preferred image having been obtained using a preferred set of imaging parameters of the plurality of sets of imaging parameters. The operations include configuring the medical imaging system to receive additional image data of the anatomical region using the preferred set of imaging parameters.

Another embodiment relates to a method. The method includes identifying, by a processing circuit of an ultrasound imaging system, contextual information regarding a patient of an ultrasound scan. The method includes determining, by the processing circuit using a machine learning model and based on the contextual information, a plurality of sets of imaging parameters. The method includes receiving, by the processing circuit, initial image data of an anatomical region obtained using the plurality of sets of imaging parameters, wherein the initial image data is obtained with an ultrasound probe in a fixed position over the anatomical region. The method includes presenting, by the processing circuit, a plurality of initial images based on the initial image data on a display device of the ultrasound imaging system, wherein each of the initial images correspond to one set of imaging parameters from among the plurality of sets of imaging parameters. The method includes receiving, by the processing circuit, a user input selecting a preferred image from among the plurality of initial images, the preferred image having been obtained using a preferred set of imaging parameters of the plurality of sets of imaging parameters. The method includes configuring the ultrasound probe to receive, by the processing circuit, additional image data of the anatomical region using the preferred set of imaging parameters.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of an artificial intelligence (AI) circuit used in the ultrasound imaging system of FIG. 1, according to an example embodiment.

FIG. 3 is a flow chart illustrating a method for providing recommended imaging parameters during an ultrasound scan using the ultrasound imaging system of FIG. 1, according to an example embodiment.

FIG. 4 is a flow chart illustrating a method for training an artificial intelligence (AI) model used during the method of FIG. 3, according to an example embodiment.

FIG. 5 is a block diagram illustrating offline training and online training of the AI model, according to an example embodiment.

FIG. 6 is a flow chart illustrating a method for collecting ultrasound data using the recommended imaging parameters provided during the method of FIG. 3, according to an example embodiment.

FIG. 7 is a block diagram illustrating a training process of the AI model during the method of FIG. 4, according to an example embodiment.

FIG. 8 is an illustration of a user interface displaying a plurality of ultrasound images each obtained using a set of recommended ultrasound imaging parameters, according to an example embodiment.

FIG. 9 is an illustration of one of the plurality of ultrasound images displayed on the user interface of FIG. 8 and the corresponding set of recommended ultrasound imaging parameters, according to an example embodiment.

FIG. 10 is an illustration of a user interface generated in response to receiving a selection of one of the plurality of ultrasound images displayed on the user interface of FIG. 8, according to an example embodiment.

### DETAILED DESCRIPTION

Referring generally to the figures, systems and methods for providing recommended medical imaging parameters are disclosed. More specifically, the systems and methods described herein include training a machine learning model to recommend medical imaging parameters based on received contextual information regarding the medical imaging procedure. For instance, the contextual information may include patient-related information, procedure-related information, operator preferences, and so on.

In existing ultrasound imaging systems that using general knowledge and user preferences, ultrasound scanners are typically preset for different applications. Then, it is up to the user to adjust the parameters for each individual case to achieve the desired image quality. In the case of an expert user, such existing systems require extra time and effort from the user to change the parameters based on the preferences and knowledge of the expert user. For an inexperienced user, however, it is not always trivial to know which parameter(s) to change and to what extent. Furthermore, incorrect imaging parameters can lead to a reduction in the image quality and the visibility of the target area.

Thus, although existing systems include preset image acquisition parameters assigned to various applications, such systems do not include the preferences of the user nor information regarding the patient that can be influential on the image quality. For example, in women's healthcare, factors such as, body mass index (BMI), age of the fetus, application, and required anatomical view can affect the image quality obtained using a specific set of acquisition parameters. Moreover, users can have different preferences for the image quality and the visibility of the internal organs in the image.

Therefore, after selecting the preset image acquisition parameters for the appropriate application using the existing technology, the expert user changes the preset image acquisition parameters in real-time during the ultrasound scan to achieve the required image quality. An inexperienced user, however, relies on the images acquired using the preset image acquisition parameters, as inexperienced users generally lack the knowledge to properly change the acquisition parameters based on patient information and do not possess the experience to develop their own preferences regarding image acquisition.

The systems and methods described herein, however, provide a technical solution to existing systems by providing a dynamic imaging parameter recommendation tool configured to obtain improved image quality during medical imaging procedures. Furthermore, by recording the usage pattern of a specific user (e.g., the changes in imaging parameters by the user in different applications), the systems described herein are configured to continuously adjust the tool such that the recommendations consider the preferences of the user as the preferences may vary with time and across applications. Unlike existing technology, the systems and methods disclosed herein provide a flexible ultrasound imaging workflow by generating multiple sets of recommended imaging parameters based on personalized patient information. That is, multiple variations of options for imaging parameters are recommended to the operator, allowing for variations among operator preferences, patient preferences, examination purposes, ultrasound protocols, etc.

The implementations described herein address a technical problem by providing enhanced data integration and analysis capabilities, which deliver a particular technical solution that streamlines and refines generation and transmittal of ultrasound images. The systems described herein are implemented to improve how data regarding an ultrasound scan is synthesized and utilized to provide high quality images based on the data. By integrating data related to a specific procedure, technician, patient, and so on, these systems provide real-time, intelligent recommendations regarding imaging parameters to use during an ultrasound scan. For example, the implementations can provide a recommendation to a sonographer based on patient information such as age, BMI, blood pressure, fetus age, etc. Accordingly, this approach provides a specific technical improvement to various technical problems, including those set forth herein.

The systems described herein may also reduce processing power by performing various processing operations simultaneously to provide intelligent recommendations regarding image acquisition parameters, rather than performing a plurality of processing operations individually and consuming unnecessary processing power. Furthermore, the systems as described herein generate recommended imaging parameters for a sonographer to use in order to execute a complete and high-quality ultrasound scan given various pieces of contextual information (e.g., sonographer preferences, patient medical history, etc.). That is, the systems as described herein are trained to identify imaging parameters required to obtain a high-quality scan in a given ultrasound procedure, therefore ensuring that the scan is complete prior to attempting to process the ultrasound images. This consideration of contextual information when generating recommended imaging parameters to use during the ultrasound scan reduces processing power by avoiding collection of unnecessary ultrasound data and submission of an incomplete and/or low-quality scan, which can cause the sonographer to have to capture additional images during a successive scan.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

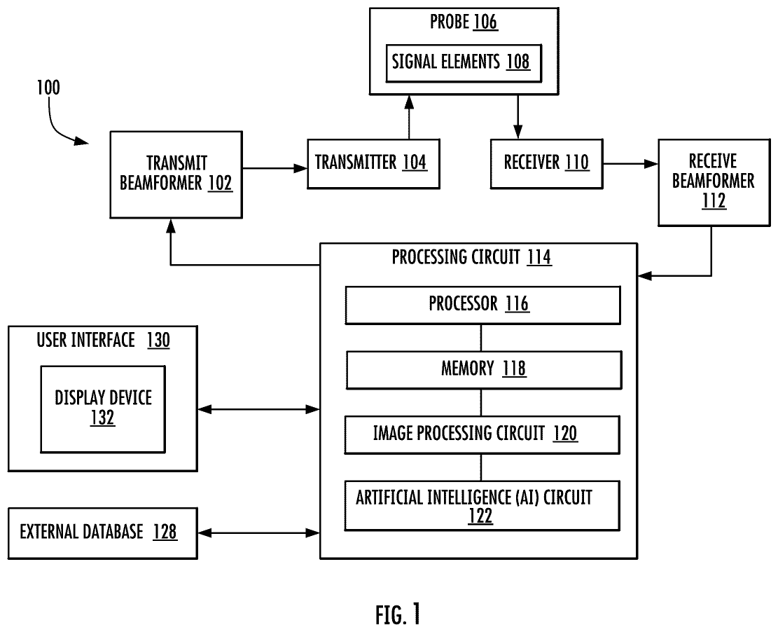

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the medical imaging parameter recommendation tool described herein may be implemented using any of a variety of medical imaging systems (e.g., medical resonance imaging, x-ray, computed tomography, positron emission tomography, etc.).

An example of a procedure performed using the ultrasound imaging system 100 may be a fetal ultrasound. The fetal ultrasound may be a first trimester ultrasound, a second trimester ultrasound, a third trimester ultrasound, or any other ultrasound scan configured to monitor and assess fetal anatomy. In some embodiments, the fetal ultrasound may be configured to determine a due date, assess a placenta size and location, confirm a normal anatomy of the fetus, detect a heartbeat, and so on. During the fetal ultrasound, a sonographer may follow a particular imaging protocol based on contextual information regarding the patient and/or the fetus (e.g., an age of the patient, an age of the fetus, patient medical history, patient BMI, patient blood pressure, patient's family health history, etc.). The contextual information may provide insight regarding the likelihood of detecting certain birth defects during the fetal ultrasound such as heart defects, spina bifida, cleft lip or palate, down syndrome, and so on. Therefore, the patient-specific imaging protocol ensures that the fetus is thoroughly captured by the ultrasound data given the contextual information and the likelihood of certain birth defects based thereon.

In at least one embodiment, the sonographer collects the ultrasound data by navigating a probe (e.g., probe 106, as described below) over the patient's uterus until a sufficient volume of ultrasound images are collected. The collected images are stored in a central storage device (e.g., memory 118) and analyzed by the sonographer. The sonographer generates a set of measurements from the images (e.g., 50-100 records), and the images and measurements are collectively reviewed by an obstetrician. The obstetrician provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a fetal skull at a first location of the probe 106. Then, the position sensor may track the movement of the probe 106 relative to the fetal skull in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a fetal examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, and an artificial intelligence (AI) circuit 122. In this way, the processing circuit 114 may be structured or configured to execute or implement the instructions, commands, and/or control processes described herein with respect to the processor 116, the memory 118, the image processing circuit 120, and the AI circuit 122. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

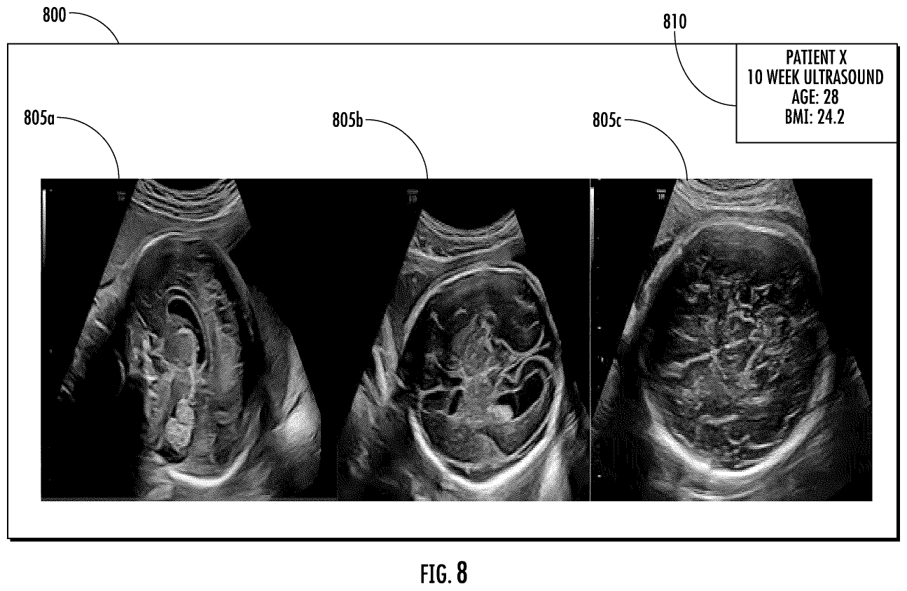

The processor 116 may also be in electronic communication with the display device 132 such that the processor 116 may process ultrasound data obtained by the probe 106 and generate images to display on the display device 132 (e.g., ultrasound images 805a-805c, as described below with reference to FIG. 8).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes'worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120 and the AI circuit 122. Both the image processing circuit 120 and the AI circuit 122 are configured to facilitate providing recommended imaging parameters during an ultrasound scan, as described herein.

The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient's uterus and the fetal anatomy contained therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

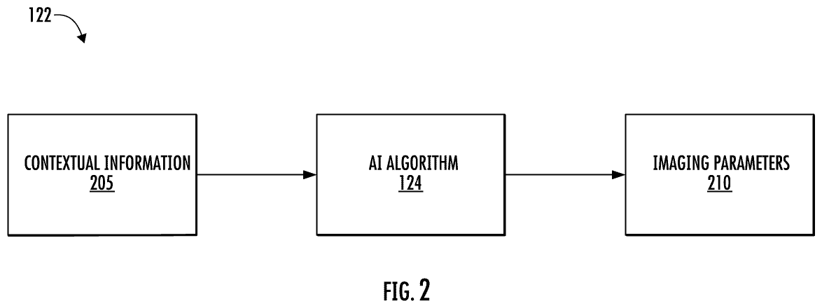

As described in greater detail below, the AI circuit 122 may be configured to recommend medical imaging parameters (e.g., imaging parameters 210, as shown in FIG. 2) based on contextual information (e.g., contextual information 205, as shown in FIG. 2) regarding a medical imaging procedure. For example, during a fetal ultrasound performed using the ultrasound imaging system 100, the AI circuit 122 may be configured to recommend at least one of a frequency, an acquisition angle, a dynamic power, a gain, or a compound imaging setting to use for image acquisition based on the age of the patient, the age of the fetus, the BMI of the patient, an application of the ultrasound imaging system 100, and so on.

The ultrasound imaging system 100 may also include an external database 128 and a user interface 130. The external database 128 refers to a database from which the processing circuit 114 (e.g., the AI circuit 122) may retrieve information used to provide recommended imaging parameters during an ultrasound scan. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the AI circuit 122 may retrieve clinical guidelines, standard practices, medical literature, medical textbooks, published research, and previous case studies related to the implementation and/or procedure. For example, if the ultrasound imaging system 100 is being used in a hospital setting to perform a first trimester fetal ultrasound on a patient with a BMI of 28.0, the AI circuit 122 may retrieve clinical guidelines and standard practices related to the hospital setting and the first trimester fetal ultrasound. Continuing with this example, the AI circuit 122 may also retrieve information from the medical literature, medical textbooks, published research, and previous case studies related to fetal anatomy and the risks associated with having a high BMI during pregnancy.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

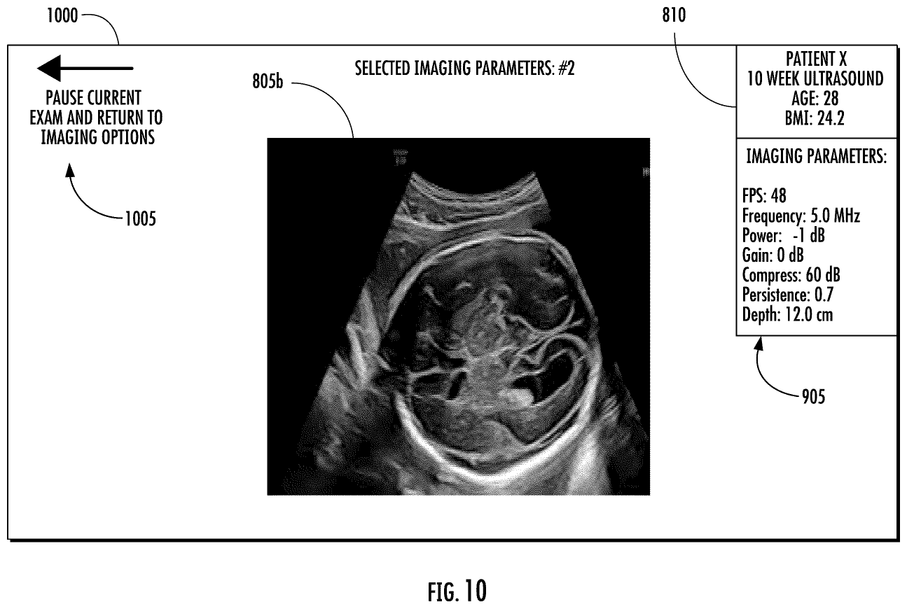

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons. As described below, FIG. 10 illustrates a GUI 1000 where at least some of the physical controls of the user interface 130 are presented by various user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring now to FIG. 2, the AI circuit 122 of the ultrasound imaging system 100 is shown in greater detail. As shown, the AI circuit 122 receives contextual information 205 regarding an ultrasound scan. In some embodiments, the contextual information 205 includes parameters regarding the patient of the ultrasound scan such as a body mass index (BMI) of the patient, an age of the patient, or a fetus age. In some instances, the contextual information 205 may be illustrated as the display of contextual information 810, as shown in FIG. 8. The contextual information 205 may include any of a plurality of factors effecting the image quality of the images generated during the ultrasound scan. For instance, in addition to the parameters regarding the patient (e.g., BMI, patient age, fetus age, etc.), the contextual information 205 may include information regarding an application of the ultrasound scan. For example, if the ultrasound scan is a fetal echocardiogram, the required image quality may differ from the required image quality of a routine first trimester fetal ultrasound in order to sufficiently capture the cardiac anatomy.

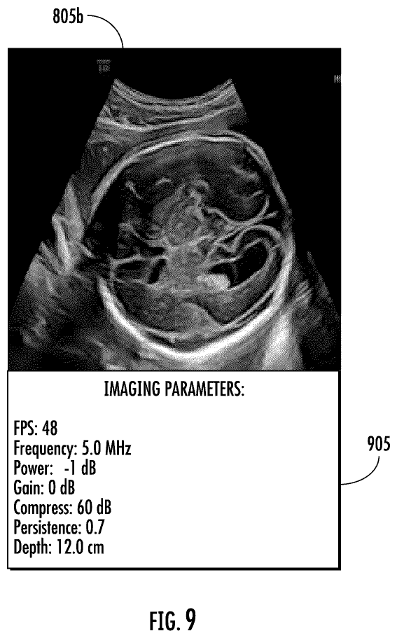

The contextual information 205 may be used as an input to an AI algorithm 124. In some embodiments, the AI algorithm 124 may be a Bayesian neural network. As shown in FIG. 2, the AI algorithm 124 is configured to generate imaging parameters 210 based on the received contextual information 205. In some instances, the imaging parameters 210 may be illustrated as the display of imaging parameters 905, as shown in FIG. 9. The imaging parameters 210 may include, for example, at least one of a frequency, an acquisition angle, a dynamic power, a gain, a compound imaging setting, and so on, to use for image acquisition during an ultrasound scan. In an example implementation of the AI circuit 122 shown in FIG. 2, if the contextual information 205 includes a BMI of 28.0, the AI algorithm 124 may recommend imaging parameters 210 that are configured to detect heart defects, neural tube defects, or any other abnormalities in the fetal anatomy that may be caused by a high BMI.

In some embodiments, the AI algorithm 124 may be trained using information regarding historic ultrasound scans performed by expert sonographers (e.g., sonographers with specific qualifications, sonographers having a number of years of experience, etc.). For example, an expert sonographer may conduct a first trimester fetal ultrasound 12 weeks into a pregnancy of a 34-year-old patient with a BMI of 28.0. The parameters regarding the first trimester fetal ultrasound (e.g., the fetus age of 12 weeks old, the patient age of 34 years old, the BMI of 28.0) may be stored (e.g., in the memory 118) as contextual information 205. Furthermore, the imaging parameters used by the expert sonographer during the first trimester fetal ultrasound may be stored (e.g., in the memory 118) as imaging parameters 210 corresponding to the contextual information 205. In some instances, the imaging parameters used by the expert sonographer refer to the acquisition parameters of an ultrasound probe (e.g., probe 106) at a moment when the image quality is accepted by the expert sonographer (e.g., when the expert sonographer instructs the ultrasound imaging system 100 to “freeze”). The AI algorithm 124 may be trained using this information regarding the historic ultrasound scan such that when the contextual information 205 regarding an ultrasound scan includes at least one of the ultrasound scan being performed at 12-weeks, the patient age being 34 years old, or the BMI being 28.0, the AI algorithm 124 may be configured to recommend imaging parameters 210 based on the imaging parameters 210 used by the expert sonographer when conducting an ultrasound scan having the same contextual information 205.

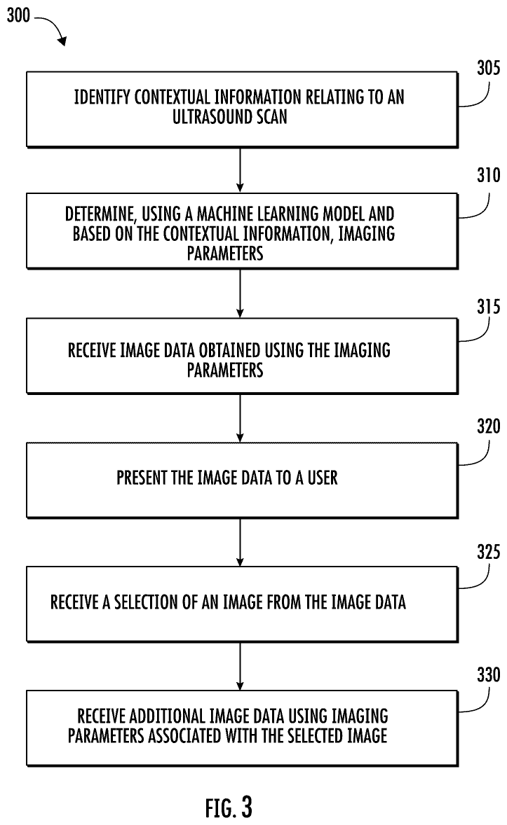

Referring to FIG. 3, a flow chart is shown illustrating a method 300 for providing recommended imaging parameters during an ultrasound scan using an ultrasound imaging system. In at least one embodiment, the ultrasound imaging system referred to by method 300 is the ultrasound imaging system 100 described above with reference to FIGS. 1 and 2, and method 300 may be implemented by the ultrasound imaging system 100. In some embodiments, method 300 may be implemented as executable instructions in a memory of the ultrasound imaging system 100, such as the memory 118 of FIG. 1.

Prior to initiating a collection of ultrasound data, method 300 may begin when an operator (e.g., a sonographer, technician, or other clinician) is authenticated as an authorized user of the ultrasound imaging system 100. In some embodiments, the operator may authenticate themselves as an authorized user of the ultrasound imaging system 100 by logging in to a portal (e.g., an online application accessible via the user interface 130) associated with the environment in which the ultrasound imaging system 100 is being implemented (e.g., a hospital or other healthcare provider). For instance, the operator may log in using a unique identifier (e.g., a username, a password, a biometric scan, a pin code, etc.).

After the operator is authenticated, the operator of the ultrasound imaging system 100 may enter patient and/or procedure-specific information into the ultrasound imaging system 100 prior to the collection of ultrasound data. For example, the operator may submit patient information (e.g., identifying information such as name, date of birth, social security number, and so on, and/or medical information such as a medical history, family medical history, a current diagnosis, and so on) via the user interface 130. In some embodiments, the operator may select the patient from a list of patients (e.g., patients associated with a scheduled procedure to be performed by the operator), and the patient information may be imported to the ultrasound imaging system 100 (e.g., from a database associated with the environment in which the ultrasound imaging system 100 is being implemented, such as a hospital). The information entered by the operator at the beginning of method 300 may include the contextual information 205 (e.g., a BMI of the patient, a patient age, a fetus age, etc.) and may therefore be used by the ultrasound imaging system 100 in determining recommended ultrasound imaging parameters, as described herein.

In addition to the patient information, the operator may enter (e.g., as a text entry) or otherwise select (e.g., from a drop-down list of procedures) the procedure that the operator is preparing to perform. For example, the operator may enter or select “echocardiogram” as the procedure. Additionally, the operator may enter or otherwise select any known pathologies or other medical conditions that may be relevant to the procedure. For instance, the operator may be performing a fetal echocardiogram on a pregnant patient in response to the pregnant patient having a high BMI, and such information may be entered into the ultrasound imaging system 100 prior to the collection of ultrasound data. In this way, the ultrasound imaging system 100 may be configured to recommend ultrasound imaging parameters, as described herein, specific to data that may be relevant to the fetal echocardiogram (e.g., sufficient imaging of the fetal heart).

As shown in FIG. 3, at step 305, method 300 may include identifying contextual information relating to an ultrasound scan being performed by the ultrasound imaging system 100. The contextual information identified at step 305 may be the contextual information 205, as described above. That is, the contextual information may include patient-related information (e.g., a BMI of the patient, patient age, fetus age, etc.) and/or procedure-related information (e.g., a type of ultrasound scan being performed, a setting in which the ultrasound scan is being performed, a sonographer performing the ultrasound scan, etc.). In some embodiments, the contextual information may be identified from the information entered by the operator of the ultrasound imaging system 100 prior to initiating the collection of ultrasound data, as described above.

At step 310, a machine learning model may be used to determine imaging parameters based on the contextual information identified at step 305. In some embodiments, step 310 may be performed by the AI circuit 122, as shown in FIG. 2. That is, at step 305, the AI algorithm 124 may be used to determine the imaging parameters 210 based on the contextual information 205. The imaging parameters determined at step 310 may refer to a plurality of sets of imaging parameters. That is, the plurality of sets of imaging parameters may include a first set of image parameters, a second set of imaging parameters, a third set of imaging parameters, and so on. For example, the first set of imaging parameters may include a rate of 48 frames per second, a frequency of 5.0 MHz, a power of −1 dB, a gain of 0 dB, a compression of 60 dB, a persistence of 0.7, and a depth of 12.0 cm, while the second set of imaging parameters may include a rate of 52 frames per second, a frequency of 5.4 MHz, a power of 0 dB, a gain of −1 dB, a compression of 55 dB, a persistence of 0.5, and a depth of 9.0 cm.

In some embodiments, as described above with reference to FIG. 2, the machine learning model used to determine the imaging parameters at step 310 may be trained using a history of interactions associated with a plurality of users (e.g., expert sonographers) of the ultrasound imaging system 100. In such embodiments, the machine learning model may be configured to identify historic image data (e.g., including a plurality of sets of imaging parameters used to obtain the historic image data) from the history of interactions associated with at least a portion of the contextual information received at step 305. Then, the machine learning model may determine the plurality of sets of imaging parameters at step 310 based on the plurality of sets of imaging parameters used to obtain the historic image data. For example, if the contextual information received at step 305 includes a BMI of 28.0, the history of interactions may include one or more ultrasound scans involving a patient BMI of 28.0. Therefore, the machine learning model may identify the imaging parameters at step 310 based on the imaging parameters used during the one or more ultrasound scans involving the patient BMI of 28.0 from the history of interactions.

Using the imaging parameters determined at step 310, image data may be obtained at step 315. In some embodiments, the image data is obtained by the transducer of the probe 106. The image data obtained at step 315 refers to image data of an anatomical region (e.g., a uterus and the fetal anatomy contained therein) using each set of imaging parameters of the plurality of sets of imaging parameters determined at step 310. That is, where step 310 includes determining the first set of imaging parameters, the second set of imaging parameters, and the third set of imaging parameters, the image data received at step 315 may include image data obtained using the first set of imaging parameters, image data obtained using the second set of imaging parameters, and image data obtained using the third set of imaging parameters.

In some instances, the image data is obtained while a user (e.g., the operator of the ultrasound imaging system 100) holds the probe 106 in a fixed position over the anatomical region for a predetermined amount of time (e.g., three to six seconds). Thus, while the probe 106 is held in the fixed position over the anatomical region, the ultrasound imaging system 100 is configured to switch between the plurality of sets of imaging parameters determined at step 310 (e.g., the first set of imaging parameters, the second set of imaging parameters, and the third set of imaging parameters) such that the image data received at step 315 includes image data obtained using each of the plurality of sets of imaging parameters determined at step 310. For example, the image data received at step 315 may refer to the ultrasound images 805a, 805b, and 805c, as shown in FIG. 8 and described in greater detail below.

At step 320, the image data received at step 315 is presented to a user. In some embodiments, the image data (e.g., ultrasound images 805a-805c) may be presented to the user via the display device 132 of the user interface 130. For example, the image data may be presented via GUI 800, as shown in FIG. 8 and described in greater detail below. In some instances, presenting the image data at step 320 includes presenting a plurality (e.g., three) of cine loops depicting the anatomical region.

According to various embodiments where the AI algorithm 124 is the Bayesian neural network, the image data may include a probability associated with each image included in the image data. For example, where the image data includes the ultrasound images 80a-805c, each of the ultrasound image 805a, the ultrasound image 805b, and the ultrasound image 805c may correspond to a probability determined by the Bayesian neural network. The probability represents a likelihood that each set of imaging parameters used to capture the respective image data (e.g., each of the ultrasound image 805a, the ultrasound image 805b, and the ultrasound image 805c) would be used by an expert sonographer during the ultrasound scan. That is, for example, ultrasound image 805a may be obtained using the first set of imaging parameters, and the Bayesian neural network may determine a corresponding probability of 70% based on the historic image data. Ultrasound image 805b may be obtained using the second set of imaging parameters, and the Bayesian neural network may determine a corresponding probability of 90% based on the historic image data. Ultrasound image 805c may be obtained using the third set of imaging parameters, and the Bayesian neural network may determine a corresponding probability of 80% based on the historic image data. Therefore, ultrasound image 805b may be obtained using a set of ultrasound imaging parameters that are more likely to be used by an expert sonographer during the ultrasound scan than each of the sets of ultrasound imaging parameters used to obtain the ultrasound image 805a and the ultrasound image 805c. In such embodiments, each of the probabilities (e.g., 70%, 90%, and 80%) may be presented at step 320 with the corresponding image data (e.g., ultrasound image 805a, ultrasound image 805b, and ultrasound image 805c, respectively).

Furthermore, in some embodiments, the Bayesian neural network may be configured to identify, from the image data received at step 315, a subset of the image data based on the probabilities associated with the image data. For example, the Bayesian neural network may be configured to identify a subset of the image data corresponding to probabilities of 70% or higher. Continuing with this example, the subset of the image data may be presented to the user at step 320 such that only image data corresponding to a probability of 70% or higher is presented to the user.

At step 325, method 300 includes receiving a selection of an image (e.g., a preferred image) from the image data presented to the user at step 320. For instance, as shown in FIG. 10, the user may select ultrasound image 805b from among the ultrasound images 805a-805c presented via the GUI 800 (e.g., as shown in FIG. 8). As another example, where step 320 includes presenting three cine loops to the user, step 325 may include receiving a selection of one of the three cine loops from the user. The user input (e.g., the selection of the image) is then stored (e.g., in memory 118) in relation to a profile corresponding to the user, as described below with reference to step 420 of method 400. In some embodiments, the selection received at step 325 may be stored via at least one of a cloud storage, the memory device (e.g., memory 118), or a server associated with an environment in which the ultrasound imaging system 100 is implemented.

Based on the selection received at step 325, additional image data is received at step 330 using the imaging parameters associated with the selected image. That is, the probe 106 may be configured to receive the additional image data from the transducer and of the anatomical region (e.g., a uterus and the fetal anatomy contained therein). In this way, the ultrasound scan continues at step 330 by collecting the additional image data using the selected set of imaging acquisition parameters (e.g., a preferred set of imaging parameters corresponding to a preferred image selected by the user). For instance, where the image selected at step 325 is ultrasound image 805b from the image data presented via GUI 800 (e.g., ultrasound images 805a-805c), the additional image data may be received using the imaging parameters included in the display of imaging parameters 905 associated with the ultrasound image 805b, as shown in FIGS. 9 and 10.

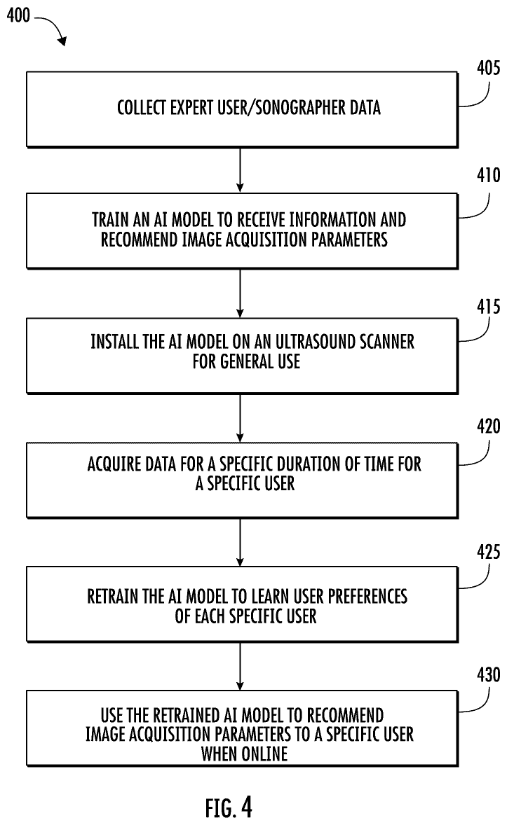

Referring to FIG. 4, a flow chart is shown illustrating a method 400 for training an AI model used during method 300. In at least one embodiment, the AI model referred to by method 400 is the AI algorithm 124 of the AI circuit 122 described above with reference to FIGS. 1 and 2, and method 400 may be implemented by the ultrasound imaging system 100. In some embodiments, method 400 may be implemented as executable instructions in a memory of the ultrasound imaging system 100, such as the memory 118 of FIG. 1.

As shown in FIG. 4, method 400 may begin with collecting expert user/sonographer data at step 405. The expert user/sonographer data collected at step 405 may include the information regarding historic ultrasound scans performed by expert sonographers, as described above with reference to FIG. 2. That is, the expert user/sonographer data may include any parameters that may affect/influence image quality during an ultrasound scan. For example, the expert user/sonographer data may include contextual information regarding the patient (e.g., age, fetus age, BMI, etc.), an application of the ultrasound scan (e.g., an echocardiogram, a first trimester fetal ultrasound scan, etc.), user preferences regarding image acquisition, and so on. The expert user/sonographer data may be collected at step 405 by the AI circuit 122 using a history of interactions associated with a plurality of users (e.g., expert users/sonographers) of the ultrasound imaging system 100 such that an AI model (e.g., the AI algorithm 124) may be pre-trained using such data (e.g., as described above with reference to FIG. 2).

At step 410, an AI model (e.g., the AI algorithm 124) is trained to receive information (e.g., the contextual information 205) and recommend image acquisition parameters (e.g., the imaging parameters 210) based on the received information. In other words, the AI model is trained at step 410 to generate outputs based on inputs as shown in FIG. 2 and described above. Furthermore, in order to provide flexibility during an ultrasound scan, the AI model may be configured to recommend a plurality of options regarding sets of imaging parameters (e.g., the imaging parameters determined at step 310 of method 300) based on the inputs (e.g., the contextual information 205). In some instances, the AI model is a Bayesian neural network configured to recommend the plurality of options. That is, rather than generating a single output (e.g., a set of imaging parameters) for a given input (e.g., contextual information regarding an ultrasound scan), the Bayesian neural network is configured to provide a statistical distribution of the output. Therefore, the Bayesian neural network generates multiple outputs (e.g., a plurality of sets of image parameters) and a probability associated with each of the multiple outputs, as described above with reference to step 320 of method 300.

After being trained to recommend the image acquisition parameters based on the received information at step 410, the AI model may be installed on an ultrasound scanner for general use at step 415. That is, the general use refers to a use of the AI model that is compatible with each user of the ultrasound imaging system 100 (e.g., not user-specific). In some embodiments, the ultrasound scanner referred to at step 415 may be the probe 106, as described above.

At step 420, data for a specific duration of time and for a specific user of the ultrasound imaging system 100 is acquired using the ultrasound scanner with the AI model installed for general use (e.g., from step 415). That is, the ultrasound imaging system 100 may identify the specific user once the user (e.g., operator, sonographer, technician, etc.) logs-in or otherwise accesses the ultrasound imaging system 100 (e.g., as described above with reference to method 300). Then, the specific user may conduct one or more ultrasound scans using the ultrasound scanner with the AI model installed for general use. In this way, the ultrasound imaging system 100 (e.g., the AI circuit 122) may monitor the user's use of the ultrasound scanner during the one or more ultrasound scans over the specific duration of time (e.g., a duration of three ultrasound scans, one month, 100 ultrasound scans, one year, etc.).

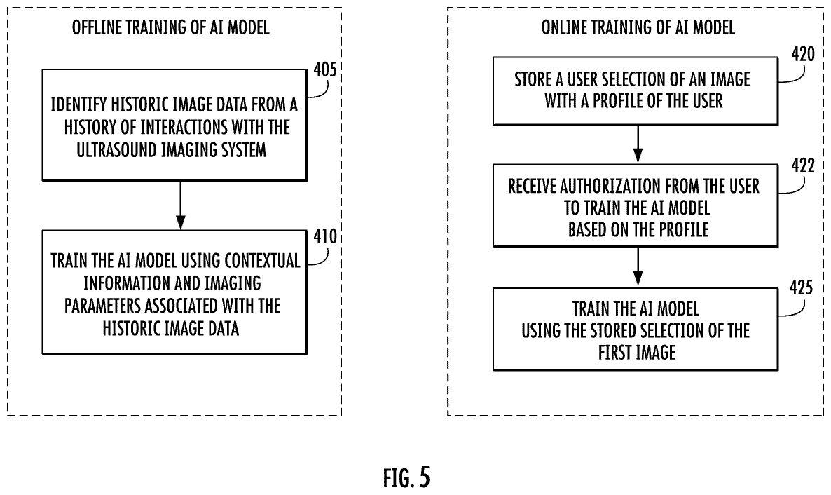

At step 425, the AI model is retrained to learn user preferences of each specific user based on the data acquired at step 420. In some embodiments, retraining the AI model to learn the user preferences of each specific user includes first receiving a permission from a specific user to retrain the AI model using the user preferences of the specific user (e.g., step 422 shown in FIG. 5). As an example, the AI circuit 122 may be configured to monitor three months of usage of the ultrasound scanner with the AI model installed for general use by the specific user. Then, after three months and with the permission of the specific user, the AI circuit 122 may be configured to retrain the AI model (e.g., the AI algorithm 124) based on the learned preferences and behavior of the specific user.

After the AI model is retrained to learn the user preferences at step 425, the retrained AI model is used at step 430 to recommend image acquisition parameters to a specific user when the specific user is online (e.g., while performing a live ultrasound scan using the ultrasound imaging system 100). That is, a profile associated with the specific user may be identified during a successive ultrasound scan and the retrained AI model may be configured to recommend image acquisition parameters based on the identified profile of the specific user. In this way, the retrained AI model may be configured to provide recommended imaging parameters during an ultrasound scan during method 300, as described above.

Referring to FIG. 5, a block diagram illustrating an offline training of the AI model and an online training of the AI model during the method of FIG. 4 is shown. That is, FIG. 5 depicts two training stages (e.g., the offline training and the online training) of the AI model during method 400 (e.g., the AI algorithm 124). In this way, the AI algorithm 124 may be configured to consider user-specific preferences when generating outputs (e.g., the imaging parameters 210, as shown in FIG. 2 and described above).

As shown in FIG. 5, the offline training of the AI model includes steps 405 and 410 of method 400. The offline training uses a dataset gathered from expert users of the ultrasound imaging system 100 to train an algorithm (e.g., the AI algorithm 124) to recommend a set of acquisition parameters. More specifically and as described above, at step 405, historic image data is identified from a history of interactions of the expert users with the ultrasound imaging system 100 (e.g., the expert/sonographer data collected at step 405 method 400 described above). Then, at step 410, the AI model is trained using contextual information (e.g., contextual information 205) and imaging parameters (e.g., imaging parameters 210) associated with the historic image data. The offline training of the AI model (e.g., steps 405 and 410 of method 400) prepares the AI model to be installed on the ultrasound scanner for general use at step 415 of method 400.

After the AI model is installed on the ultrasound scanner for general use at step 415, the online training of the AI model is shown to include steps 420 and 425 of method 400. As described above, the data is acquired for a specific duration of time for a specific user at step 420. Furthermore, and as shown in FIG. 5, step 420 may include storing a user selection of an image with a profile of the user (e.g., the selection of the image from step 325 of method 300). That is, the user selection of the image may be acquired during the specific duration of time and stored with the profile of the specific user. In some embodiments, the user selection may be stored via at least one of a cloud storage, the memory device (e.g., memory 118), or a server associated with an environment in which the ultrasound imaging system is implemented.

FIG. 5 also includes receiving an authorization (e.g., permission) from the user to train the model based on the profile at step 422. That is, as described above with reference to FIG. 4, retraining the AI model to learn the user preferences of each specific user may include first receiving a permission from a specific user to retrain the AI model based on the user preferences of the specific user. After receiving the authorization at step 422, the online training includes training the AI model at step 425 using the stored selection of the image (e.g., the selection of the image from step 325 of method 300). In this way, the online training includes retraining the AI model to learn user preferences of each specific user. In sum, the offline training is configured to train the AI model for general (e.g., not user-specific) use on a large dataset of historical interactions with the ultrasound imaging system 100. Then, the online training accounts for learned user preferences such that the AI model can provide more accurate outputs (e.g., imaging parameters 210) depending on the specific user of the ultrasound imaging system 100.

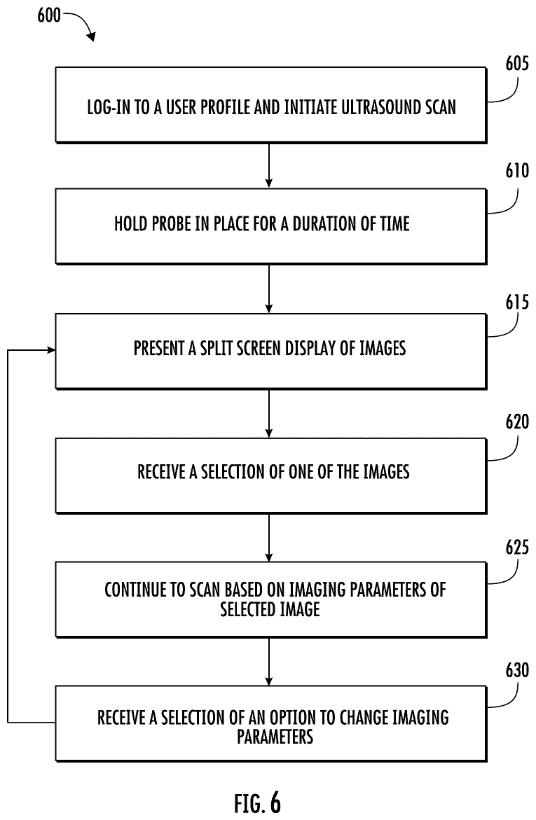

Referring to FIG. 6, a flow chart is shown illustrating a method 600 for collecting ultrasound data using the recommended imaging parameters provided during method 300, as described above with reference to FIG. 3. In at least one embodiment, the ultrasound imaging system referred to by method 600 is the ultrasound imaging system 100 described above with reference to FIGS. 1 and 2, and method 600 may be implemented by the ultrasound imaging system 100. In some embodiments, method 600 may be implemented as executable instructions in a memory of the ultrasound imaging system 100, such as the memory 118 of FIG. 1.

As shown in FIG. 6, method 600 may begin when a user logs-in to a user profile and initiates an ultrasound scan at step 605. Furthermore, step 605 may include loading contextual information 205, such as information relating to sonographer preferences, patient information, procedure information, etc.

At step 610, an ultrasound probe (e.g., the probe 106) may be held in place for a duration of time. In some embodiments, the user may hold the probe 106 in a stationary position over an anatomical region being imaged during the ultrasound scan. For example, during a fetal examination, the user may hold the probe 106 in a stationary position over a patient's uterus for three to six seconds. In some embodiments, the image data collected while the ultrasound probe is held in place at step 610 may be the image data received at step 315 of method 300 using the imaging parameters determined at step 310. That is, as described above, the ultrasound imaging system 100 automatically changes AI-recommended sets of imaging parameters and acquires images/cine according to each of the sets of imaging parameters. For example, the set of imaging parameters being used to collect the ultrasound data may change twice after each 1-2 second interval in order to generate three cine loops, each 1-2 seconds long and each obtained using a distinct set of imaging parameters.

After the probe is held in place for the duration of time at step 610, a split-screen display of images is presented to the user at step 615. For instance, the split screen display may be the GUI 800, as shown in FIG. 8, and the images displayed thereon may be ultrasound images 805a-805c. In this way, the split-screen display presented at step 615 allows the user to perform a side-by-side comparison of each ultrasound image/cine obtained using a respective set of imaging parameters.

At step 620, a selection of one of the images presented via the split-screen display at step 615 is received. In some embodiments, the selection received at step 620 of method 600 refers to the selection received at step 325 of method 300, as described above with reference to FIG. 3. For example, the selection may be a selection of ultrasound image 805b from among the ultrasound images 805a-805c, as shown in FIG. 10.

Based on the selection received at step 620, the ultrasound scan continues at step 625 based on imaging parameters of the selected image. Continuing with the example of selecting image 805b, the ultrasound scan may continue at step 625 using the imaging parameters 905, as shown in FIGS. 9 and 10.

At step 630, a selection of an option to change the imaging parameters (e.g., the imaging parameters used during the continuation of the ultrasound scan at step 625) is received. In some embodiments, the option selected at step 630 refers to selectable element 1005, as shown in FIG. 10. As described below with reference to FIG. 10, the selectable element 1005 may be configured to present the split screen (e.g., the GUI 800) once selected. In this way, after receiving the selection of the option to change the imaging parameters at step 630, method 600 may return to step 615, where the split screen display of images is presented to the user.

Therefore, method 600 may represent an iterative process, with steps 615 through 625 being repeated upon receiving the selection of the option to change the imaging parameters at step 630. For example, in a first iteration of method 600, the selection received at step 620 may be a selection of image 805b and step 625 may include continuing the ultrasound scan using the imaging parameters associated with the ultrasound image 805b. During a second iteration of method 600 (e.g., upon receiving the split-screen display of ultrasound images 805a-805c at step 615 after selecting the option to change the imaging parameters), however, the selection received at step 620 may be a selection of image 805a and step 625 may include continuing the ultrasound scan using the imaging parameters associated with the ultrasound image 805a.

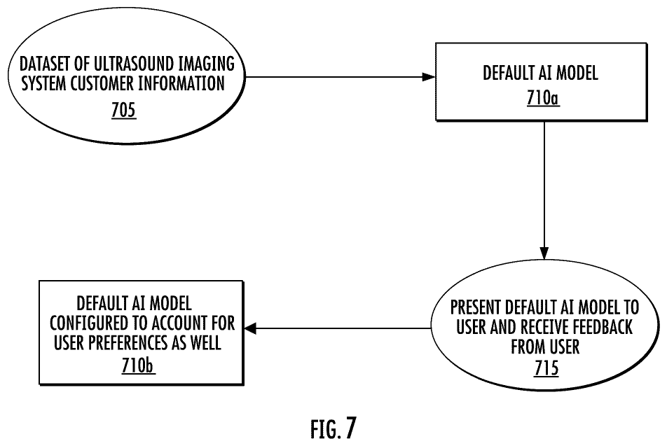

Referring to FIG. 7, a block diagram illustrating the training of the AI model during method 400 of FIG. 4 is shown. More specifically, FIG. 7 depicts a dataset 705 of ultrasound imaging system customer information (e.g., information regarding users of the ultrasound imaging system 100). In some embodiments, the dataset 705 may include the information collected at step 405 of method 400. The dataset 705 may be used to train a default AI model 710a, as described above with reference to step 410 of method 400. Therefore, the dataset 705 and the default AI model 710a of FIG. 7 may represent the offline training of the AI model, as described above with reference to FIG. 5.

At block 715, the default AI model 710a is presented to a user and feedback is received from the user. In order words, block 715 may represent installing the default AI model 710a on a scanner for general use, such as at step 415 of method 400. Receiving the feedback from the user at block 715 may include storing a selection of an image (e.g., the selection of the image received at step 325 of method 300) recommended by the default AI model 710a, as described above with reference to step 420 of method 400. The received feedback may then be used to configure the default AI model 710a such that the default AI model 710a is retrained to account for user preferences at step 710b, and as described above with reference to retraining the AI model at step 425 of method 400. In this way, blocks 715 and 710b may represent the online training of the AI model, as described above with reference to FIG. 5.

Referring to FIG. 8, a GUI 800 displaying a plurality of ultrasound images 805a, 805b, and 805c is shown. In some embodiments the GUI 1000 may be a GUI generated for display on the display device 132. Further, the GUI 1000 may be configured as a touch screen display, such that the user may engage with the information contained thereon by touching the respective location of the information on the touch screen display. For example, each of the ultrasound images 805a, 805b, and 805c may be configured as a selectable element such that the user can select one of the ultrasound images 805a, 805b, and 805c by touching the respective location of the ultrasound image on the touch screen display.

As described above, the GUI 800 may be the split-screen display presented at step 615 of method 600. Furthermore, each of the plurality of ultrasound images 805a, 805b, and 805c may be obtained using a set of recommended ultrasound imaging parameters determined at step 310 of method 300 and thus may be the image data presented to the user at step 320 of method 300. The GUI 800 is also shown to include the display of contextual information 810 (e.g., patient X, 10-week ultrasound, age 28, BMI: 24.2). The display of contextual information 810 may include contextual information 205 received as an input by the AI algorithm 124 (e.g., the contextual information identified at step 305 of method 300).

Referring to FIG. 9, an illustration of one of the plurality of ultrasound images displayed on the user interface of FIG. 8 and the corresponding set of recommended ultrasound imaging parameters (e.g., imaging parameters 210) is shown. More specifically, FIG. 9 depicts the ultrasound image 805b and the display of imaging parameters 905 used to obtain the ultrasound image 805b (e.g., FPS: 48, frequency: 5.0 MHz, power: −1 dB, gain: 0 dB, compression: 60 dB, persistence: 0.7, depth: 12.0 cm). The display of imaging parameters 905 may refer to a set of imaging parameters determined at step 310. In some embodiments, as described above, ultrasound image 805b may be selected from among the plurality of ultrasound images 805a, 805b, and 805c presented via the GUI 800 at step 320 of method 300.

Referring to FIG. 10, a GUI 1000 generated in response to receiving a selection of one of the plurality of ultrasound images 805a, 805b, and 805c displayed on GUI 800 is shown. As shown in FIG. 10, the selected image from GUI 800 is ultrasound image 805b. The GUI 1000 is also shown to include the display of contextual information 810 and the display of imaging parameters 905 associated with the selected image (e.g., the imaging parameters associated with ultrasound image 805b, as shown in FIG. 9). Therefore, based on the selection of image 805b, the ultrasound imaging system 100 is configured to collect more image data of the anatomical region depicted in ultrasound image 805b using the parameters included in the display of parameters 905.

The GUI 1000 also includes selectable element 1005, which represents an option to pause the current ultrasound scan (e.g., exam) and return to imaging options. That is, upon receiving a selection of the selectable element 1005 (e.g., as described above with reference to step 630 of method 600), the ultrasound imaging system 100 is configured to present the split-screen shown in FIG. 8 such that the user can select another ultrasound image from the ultrasound images 805a, 805b, 805c. As described above with reference to FIG. 6, the iterative process may continue with obtaining new image data using a different set of imaging parameters associated with a new selection from among the ultrasound images 805a, 805b, 805c.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. §112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit and receive an ultrasound signal;
a matching layer configured to have an acoustic impedance between a tissue to be imaged and a material of the transducer;
a damping block configured to absorb ultrasound energy; and
a processing circuit comprising a processor coupled to a memory device storing instructions thereon that, when executed, cause the processing circuit to perform operations comprising:
identifying contextual information regarding a patient of an ultrasound scan;
determining, using a machine learning model and based on the contextual information, a plurality of sets of imaging parameters;
receiving initial image data obtained by the transducer and of an anatomical region obtained using the plurality of sets of imaging parameters, wherein the initial image data is obtained with an ultrasound probe in a fixed position over the anatomical region;
presenting a plurality of initial images based on the initial image data on a display device of the ultrasound imaging system, wherein each of the initial images correspond to one set of imaging parameters from among the plurality of sets of imaging parameters;
receiving a user input selecting a preferred image from among the plurality of initial images, the preferred image having been obtained using a preferred set of imaging parameters of the plurality of sets of imaging parameters; and
configuring the ultrasound probe to receive additional image data from the transducer and of the anatomical region using the preferred set of imaging parameters.

2. The ultrasound imaging system of claim 1, wherein the operations further comprise storing the user input selecting the preferred image in relation to a profile of a user.

3. The ultrasound imaging system of claim 2, wherein the user input is stored via at least one of a cloud storage, the memory device, or a server associated with an environment in which the ultrasound imaging system is implemented.

4. The ultrasound imaging system of claim 2, wherein the operations further comprise training the machine learning model based on the user input selecting the preferred image.

5. The ultrasound imaging system of claim 4, wherein the plurality of sets of imaging parameters is a first plurality of sets of imaging parameters, the initial image data is first initial image data, and the operations further comprise:
identifying the profile of the user during a successive ultrasound scan;
determining, by the trained machine learning model, a second plurality of sets of imaging parameters; and
receiving second initial image data obtained using the second plurality of sets of imaging parameters during the successive ultrasound scan.

6. The ultrasound imaging system of claim 1, wherein the contextual information includes at least one of a body mass index (BMI) of the patient, an age of the patient, or a fetus age.

7. The ultrasound imaging system of claim 1, wherein the machine learning model comprises a Bayesian neural network.

8. The ultrasound imaging system of claim 7, wherein the operations further comprise:
identifying, using the Bayesian neural network, a probability associated with each of the plurality of initial images; and
presenting a subset of the plurality of initial images on the display device based on the probability associated with each of the plurality of initial images.

9. The ultrasound imaging system of claim 1, wherein the machine learning model is pre-trained using a history of interactions associated with a plurality of users of the ultrasound imaging system, and wherein the operations further comprise:
identifying historic image data from the history of interactions associated with at least a portion of the contextual information; and
determining, using the machine learning model, the plurality of sets of imaging parameters based on the plurality of sets of imaging parameters being used to obtain the historic image data.

10. The ultrasound imaging system of claim 1, wherein the plurality of sets of imaging parameters comprises at least one of a frequency, an acquisition angle, a dynamic power, a gain, or a compound imaging setting.

11. The ultrasound imaging system of claim 1, wherein the user input is a first user input, the preferred image is a first preferred image, and upon receiving the additional image data using the preferred set of imaging parameters, the operations further comprise:
receiving a second user input requesting to view the plurality of initial images;
presenting the plurality of initial images on the display device;
receiving a third user input selecting a second preferred image from the plurality of initial images, wherein the second preferred image differs from the first preferred image and corresponds to a second set of imaging parameters of the plurality of imaging parameters; and
configuring the ultrasound probe to receive updated additional image data using the second set of imaging parameters.

12. A medical imaging system comprising:
a processing circuit having a processor coupled to a memory device storing instructions thereon that, when executed, cause the processing circuit to perform operations comprising:
identifying contextual information regarding a patient of a medical imaging scan;
determining, using a machine learning model and based on the contextual information, a plurality of sets of imaging parameters;
receiving initial image data of an anatomical region obtained using the plurality of sets of imaging parameters, wherein the initial image data is obtained with an ultrasound probe in a fixed position over the anatomical region;
presenting a plurality of initial images based on the initial image data on a display device of the medical imaging system, wherein each of the initial images correspond to one set of imaging parameters from among the plurality of sets of imaging parameters;
receiving a user input selecting a preferred image from among the plurality of initial images, the preferred image having been obtained using a preferred set of imaging parameters of the plurality of sets of imaging parameters; and
configuring the medical imaging system to receive additional image data of the anatomical region using the preferred set of imaging parameters.

13. The medical imaging system of claim 12, wherein the medical imaging system is an ultrasound imaging system comprising:
a transducer configured to transmit and receive an ultrasound signal;
wherein the initial image data and the additional image data are obtained by the transducer.

14. The medical imaging system of claim 12, wherein the plurality of sets of imaging parameters is a first plurality of sets of imaging parameters, the initial image data is first initial image data, and the operations further comprise:
storing the user input selecting the preferred image in relation to a profile of a user;
training the machine learning model based on the user input selecting the preferred image;
identifying the profile of the user during a successive medical imaging scan;
determining, by the trained machine learning model, a second plurality of sets of imaging parameters; and
receiving second initial image data obtained using the second plurality of sets of imaging parameters during the successive medical imaging scan.

15. The medical imaging system of claim 12, wherein the machine learning model comprises a Bayesian neural network and wherein the operations further comprise:
identifying, using the Bayesian neural network, a probability associated with each of the plurality of initial images; and
presenting a subset of the plurality of initial images on the display device based on the probability associated with each of the plurality of initial images.

16. A method comprising:
identifying, by a processing circuit of an ultrasound imaging system, contextual information regarding a patient of an ultrasound scan;
determining, by the processing circuit using a machine learning model and based on the contextual information, a plurality of sets of imaging parameters;
receiving, by the processing circuit, initial image data of an anatomical region obtained using the plurality of sets of imaging parameters, wherein the initial image data is obtained with an ultrasound probe in a fixed position over the anatomical region;
presenting, by the processing circuit, a plurality of initial images based on the initial image data on a display device of the ultrasound imaging system, wherein each of the initial images correspond to one set of imaging parameters from among the plurality of sets of imaging parameters;
receiving, by the processing circuit, a user input selecting a preferred image from among the plurality of initial images, the preferred image having been obtained using a preferred set of imaging parameters of the plurality of sets of imaging parameters; and
configuring the ultrasound probe to receive, by the processing circuit, additional image data of the anatomical region using the preferred set of imaging parameters.

17. The method of claim 16, wherein the method further comprises:
storing, by the processing circuit, the user input selecting the preferred image in relation to a profile of a user; and
training, by the processing circuit, the machine learning model based on the user input selecting the preferred image.

18. The method of claim 17, wherein the plurality of sets of imaging parameters is a first plurality of sets of imaging parameters, the initial image data is first initial image data, and the method further comprises:
identifying, by the processing circuit, the profile of the user during a successive ultrasound scan;
determining, by the processing circuit using the trained machine learning model, a second plurality of sets of imaging parameters; and
receiving, by the processing circuit, a second initial image data obtained using the second plurality of sets of imaging parameters during the successive ultrasound scan.

19. The method of claim 16, wherein the machine learning model comprises a Bayesian neural network and wherein the method further comprises:
identifying, by the processing circuit, using the Bayesian neural network, a probability associated with each of the plurality of initial images; and
presenting, by the processing circuit, a subset of the plurality of initial images on the display device based on the probability associated with of the plurality of initial images.

20. The method of claim 16, wherein the machine learning model is pre-trained using a history of interactions associated with a plurality of users of the ultrasound imaging system, and wherein the method further comprises:
identifying, by the processing circuit, historic image data from the history of interactions associated with at least a portion of the contextual information; and
determining, by the processing circuit using the machine learning model, the plurality of sets of imaging parameters based on the plurality of sets of imaging parameters being used to obtain the historic image data.
